SEQUENCE LISTING (10 sequences)

SEQ ID NO: 1 - DNA, 1113 bp, Mucor circinelloides sp.
atgggcgaaaaattaaaacgcaagagcctcaaagaggtggtaaggcactttacaccgtcatggtttagtgttattatgggaacaggcattctgtcaattctgctgcacgtctttcctttccaattccgagggctgcaaacaattgcgctggttgtgtacattatgaatgttgtcatgttctgcatatttttgattgtcagcattgcaagatataccatctggccatccatcattcgactggtgctggagcattcaaatcagagtctatttatcggaaccatgccaatgggcttaacaaccatcaccaatttcacaacgcttgtcattgttgataaattcgcctggggtctagatctagcatttgtactgtggatcattgaatacgtattgactatagcaacagttctggttgtgccctactttgttattgtgcatcacaatcatgcattggagtctatgaacggcacttggctgctgcccattgtcccatgtgtggtcgcttctgcttcgggtggcttactggctcaatatctggatcaaggtcgtgctgtggtcgtcttgttcatatctattattaccatggggatgggcttattattagccttttctgtcattgtgatttatttttatcgtttggtggtacacaaactgccgcccagagaagtaatcattagttcctttttgccattaggtccactgggacaaggtgcgtatggtgtgattcagttgggcattgcaagtaaaacagtgttgggcgatcgatacattgcgggcttgggcgacgttgctcattctgtgggattccttatggctctatttctgtggggttacggtatttggtttttggtagtagctaccttctctgtaggtattacgacaaagcaaggtattcccttcatatgggctggtgggctttaactttcccattgggcgtatttactactgctaccttaagtattggcaacatcctcgactccatgttctttttggtattgggagccatctttacatgcatgttagttttgatatggttggccgtcatggcaaaaacactgaagggaatatttactggcgaaatgttttatgctccttgcttatctcctgtaactttaa

SEQ ID NO: 2 - protein, 371 aa, Mucor circinelloides sp.
MGEKLKRKSLKEVVRHFTPSWFSVIMGTGILSILLHVFPFQFRGLQTIALVVYIMNVVMFCIFLIVSIARYTIWPSIIRLVLEHSNQSLFIGTMPMGLTTITNFTTLVIVDKFAWGLDLAFVLWIIEYVLTIATVLVVPYFVIVHHNHALESMNGTWLLPIVPCVVASASGGLLAQYLDQGRAVVVLFISIITMGMGLLLAFSVIVIYFYRLVVHKLPPREVIISSFLPLGPLGQGAYGVIQLGIASKTVLGDRYIAGLGDVAHSVGFLMALFLWGYGIWFLVVATFSVGITTKQGIPFNMGWWALTFPLGVFTTATLSIGNILDSMFFLVLGAIFTCMLVLIWLAVMAKTLKGIFTGEMFYAPCLSPVTL

SEQ ID NO: 3 - DNA, 51 bp, Artificial sequence
  MuC4mt-F
acttttatatacaaaataactaaatctcgagatgggcgaaaaattaaaacg

SEQ ID NO: 4 - DNA, 46 bp, Artificial sequence
  MuC4mt-R
actagtcgcaattgccgcggctcgagctacagagaaggtagagaat

SEQ ID NO: 5 - DNA, 29 bp, Artificial sequence
  1552-F
cctcggcgtcatgatgtttttgtgtacct

SEQ ID NO: 6 - DNA, 27 bp, Artificial sequence
  1552-R
gggatgtctgctgctaccatgtctcat

SEQ ID NO: 7 - DNA, 32 bp, Artificial sequence
  WJ11-c4mt-F
ttatttttatcgtttggtggtacacaaactgc

SEQ ID NO: 8 - DNA, 28 bp, Artificial sequence
  WJ11-c4mt-R
gtaaccccacagaaatagagccataagg

SEQ ID NO: 9 - DNA, 19 bp, Artificial sequence
  actin-F
gatgaagcccaatccaaga

SEQ ID NO: 10 - DNA, 19 bp, Artificial sequence
  actin-R
ttctcacggttggacttgg

(19) United States
(12) Patent Application Publication
(10) Pub. No.: US 2022/0177526 A1
(43) Pub. Date: Jun. 9, 2022

(54) C4-DICARBOXYLIC ACID TRANSPORTER FOR INCREASING OIL YIELD OF MUCOR CIRCINELLOIDES

(71) Applicant: SHANDONG UNIVERSITY OF TECHNOLOGY, Zibo (CN)

(72) Inventors: Yuanda SONG, Zibo (CN); Junhuan YANG, Zibo (CN); Wu YANG, Zibo (CN)

(73) Assignee: SHANDONG UNIVERSITY OF TECHNOLOGY, Zibo (CN)

(21) Appl. No.: 17/270,661

(22) PCT Filed: Nov. 20, 2019
(86) PCT No.: PCT/CN2019/119646

§ 371 Date: Feb. 23, 2021

(30) Foreign Application Priority Data
Nov. 23, 2018 (CN) 201811402944.6

(51) Int. Cl. C07K 14/37; C12N 15/80

(52) U.S. Cl. CPC C07K 14/37; C12N 15/80

(57) ABSTRACT

A C4-dicarboxylic acid transporter and its encoding gene C4mt gene can increase oil yield of Mucor circinelloides, the C4mt gene may be cloned from the high-yield M. circinelloides WJ11, and the C4mt gene is transformed into M. circinelloides deficient strain Mu402, the C4mt gene can be integrated into the genome of M. circinelloides by homologous recombination to obtain recombinant strain Mu-C4mt. The total fatty acid content of the Mu-C4mt strain can be increased by 25.30% and the intracellular lipid content may reach up to 16.34% of the dry biomass.

### SEQUENCE LISTING

The instant application contains a Sequence Listing which has been submitted electronically in ASCII format and is hereby incorporated by reference in its entirety. Said ASCII copy, created on Jun. 4, 2021, is named 534665USSL.txt and is 6,628 bytes in size.

### CROSS REFERENCE TO RELATED APPLICATION

This application is a national stage application under 35 USC § 371 of International Appl. PCT/CN2019/119646, filed on Nov. 20, 2019, which claims the priority of Chinese Patent Application No. 201811402944.6 entitled “C4-dicarboxylic acid transporter for increasing oil yield of Mucor circinelloides” filed with China National Intellectual Property Administration on Nov. 23, 2018, the content of each of which is incorporated herein by reference in its entirety.

### TECHNICAL FIELD

The present application belongs to the technical field of genetic engineering, especially relating to a C4-dicarboxylic acid transporter for increasing oil yield of Mucor circinelloides.

### BACKGROUND ART

Oleaginous microorganisms can synthesize triglycerides in large quantities, among which filamentous fungi and microalgae can synthesize long-chain polyunsaturated fatty acids, and these biologically active fatty acids have been recognized as important nutritional food resources. With the change of people's living standards, increasingly individuals pay consideration to their health and the quality of life. The dietary fatty acids and nutrition health have become hotspots of chronic disease studies. The important active polyunsaturated fatty acids in the oil, such as γ-linolenic acid (GLA). α-linolenic acid (ALA), lithospermic acid (stearidonic acid, SDA), eicosapentaenoic acid (EPA) and docosahexaenoic acid (DHA), maintains lipid metabolism in vivo and prevent the occurrence of various chronic diseases. γ-linolenic acid needs to be obtained through the diet is an essential fatty acid for the human body which is the precursor to synthesize many active polyunsaturated fatty acids. The oleaginous microorganisms have attracted much attention due to the characteristics of high oil content, short growth cycle, wide spectrum utilization of carbon source and the like, and the polyunsaturated fatty acids produced by microorganisms have been used as dietary supplements that are being accepted by people. With the development of technologies such as fermentation, extraction, and processing of microbial oils, the oleaginous microorganisms will play an important role in the future biological oil production industry.

M. circinelloides is the first commercial cultured strain for producing oils and fats by utilizing microorganisms in the world. M. circinelloides, as an oleaginous fungus, has been used as a model organism to investigate the mechanism of lipid accumulation. However, a new strain M. circinelloides WJ11 was isolated in this study, which produces lipids that may account for 36% of the dry cell weight, its genome sequencing is completed. The research on the genetic background and the oil production mechanism is conducted in-depth, and meanwhile, its genetic manipulation thereof is simple, various genetic tools are available, so that it is more suitable for preparation of cell factories. M. circinelloides produces a large amount of γ-linolenic acid (GLA), which has important physiological functions in human body and this is its main commercial value.

The C4-dicarboxylic acid transporter (C4-dicarboxylate/malic acid transporter, (C4mt) gene is one of the key factors in lipid synthesis. Under the condition of sufficient carbon source and lack of other nutrients (such as nitrogen, phosphorus, sulfur, etc.), the final product pyruvate of glycolysis enters mitochondria, the tricarboxylic acid cycle in mitochondria is blocked, and resulting a large amount of citric acid accumulation in mitochondria. At this time, citric acid is transported to the cytoplasm and is cleaved by a citrate acid lyase to produce acetyl coenzyme A as fatty acids synthesis substrates and oxaloacetate. Acetyl coenzyme A is a precursor substance for synthesizing oil and fat in cells, and fatty acids are stored in cells in the form of triglycerides. Therefore, the transfer of citric acid plays an important role in the accumulation of microbial cell oil. It has been reported that a C4-dicarboxylic acid transporter can transport malic acid and other dicarboxylic acids in the cytoplasm from mitochondria through the mitochondrial cell membrane, thereby promoting the transport of citric acid and promoting the synthesis of cellular oils and fats. Therefore, the C4-dicarboxylic acid transporter plays an important role in the synthesis and accumulation of microbial oils and fats.

### SUMMARY OF THE INVENTION

The purpose of the present application is to provide a C4-dicarboxylic acid transporter for increasing oil yield of M. circinelloides.

To achieve the above objective, the present application provides the following schemes:

The present application provides a C4-dicarboxylic acid transporter for increasing the oil yield of M. circinelloides, the amino acid sequence of the C4-dicarboxylic acid transporter is set forth in SEQ ID NO:2.

The application also provides a C4mt gene for coding the C4-dicarboxylic acid transporter, and the nucleotide sequence of the C4mt gene is set forth in SEQ ID NO. 1.

The application also provides a recombinant vector containing the C4mt gene.

In some embodiments, the recombinant vector can express the C4-dicarboxylic acid transporter of M. circinelloides, and the vector is an expression vector of M. circinelloides.

In some embodiments, pMAT1552 is an original vector to obtain recombinant vector.

The application also provides a transformant containing the recombinant vector in the above scheme.

In some embodiments, the transformant can express the C4-dicarboxylic acid transporter of M. circinelloides.

In some embodiments, M. circinelloides is a host strain of the recombinant vector.

In some embodiments, the M. circinelloides strain includes a M. circinelloides deficient strain Mu402.

The present application also provides a use of the C4-dicarboxylic acid transporter or the C4mt gene or the recombinant vector or the recombinant M. circinelloides for increasing the oil yield.

The technical scheme of the application is as follows: extracting mRNA of M. circinelloides WJ11 strain to be reverse transcribed to cDNA, designing specific primers to amplify C4-dicarboxylic acid transporter (C4mt) gene by PCR and linking the gene to integrative plasmid pMAT1552, then electrically transforming the recombinant vector into protoplast of M. circinelloides deficient strain Mu402, selecting positive clones for fermentation culture, wherein the fermentation conditions are as follows: using Kendrick culture medium, 28° C., 700 rpm, air intake 1 v/vmin⁻¹, pH 6.0. During the fermentation process, collecting samples according to oil accumulation law, and determining the oil content and composition.

The beneficial effects of the present application: the present application provides a C4-dicarboxylic acid transporter for increasing the oil yield of M. circinelloides, and the transformant strain Mc-C4mt constructed by using the gene encoding the C4-dicarboxylic acid transporter. Compared with the control strain Mc1552, the yield of the intracellular lipid produced by the recombinant strain Mc-C4mt is increased by 25.30%, and the content of the intracellular lipid can reach 16.34% of the dry biomass. The present application uses M. circinelloides as a model strain for studying oil producing cell factories, by utilizing the genetic engineering method. The present application also provides direction for popularizing the industrial application of the M. circinelloides, that produces polyunsaturated fatty acids with high nutritional value, which meets the growing requirements of people on body health and high-quality life.

### BRIEF DESCRIPTION OF THE DRAWINGS

FIG. 1 is a PCR spectrum for identification of recombinant strain of Mucor circinelloides, where M represents standard nucleic acid molecular weight; 0 represents a control strain Mc1552; 1-3 represents recombinant strain Mc-C4mt of Mucor circinelloides;

FIG. 2 is a graph showing the determination of the mRNA expression level of the C4mt gene of the recombinant strain of Mucor circinelloides.

### DETAILED DESCRIPTION OF THE EMBODIMENTS

The present application will be further described below with reference to examples.

### Example 1 Informatics Analysis of C4-Dicarboxylic Acid Transporter (C4mt) Gene

According to the genome information of the sequenced WJ11, a C4-dicarboxylic acid transporter (C4mt) gene (000 18.48, 1113 bp) (the nucleotide sequence of which was set forth in SEQ ID NO: 1) was found, and the gene sequence was used for informatics analysis. The coding region of this sequence could code 370 amino acids (the sequence of the amino acids as set forth in SEQ ID NO: 2), and the predicted molecular weight was 32.12 kDa and PI was 9.16, and the protein coded by the sequence had homology of 91% and 67% respectively with a C4-dicarboxylic acid transporter gene (NCBI gene ID: GAN00662.1) from Mucor ambiguuss and a C4-dicarboxylic acid transporter gene (NCBI gene ID: XP_023469779.1) from Rhizopus microsporus ATCC 52813 so that it is preliminarily determined that the gene could code the C4-dicarboxylic acid transporter (C4mt) of M. circinelloides WJ11.

### Example 2: Construction of Recombinant Vector

The M. circinelloides WJ11 strain was inoculated into a 500 mL baffled flask which contained 100 mL of Kendrick medium (glucose 30 g/L, MgSO₄.7H₂O 1.5 g/L, ammonium tartrate 3.3 g/L, KH₂PO₄ 7.0 g/L, Na₂HPO₄ 2.0 g/L, yeast extract 1.5 g/L, CaCl₂ 0.076 g/L, FeCl₃.6H₂O 8 mg/L, ZnSO₄.7H₂O 1 mg/L, CuSO₄.5H₂O 0.1 mg/L, Co(NO₃)₂.6H₂O 0.1 mg/L. MnSO₄.5H₂O 0.1 mg/L), cultured at 28° C., 150 rpm, for 24 h, the samples were collected by suction filtration, and DNA was extracted, cDNA was reverse transcribed. According to the genome information of sequenced WJ11, the C4-dicarboxylic acid transporter (C4mt) gene (scaffold00018.48,1113 bp) was found (the nucleotide sequence was wet forth in SEQ ID NO:1), and the specific primer MuC4mt-F and MuC4mt-R were designed according to the gene sequence, the M. circinelloides cDNA was used as template for PCR amplification, MuC4mt-F was set forth in SEQ ID NO:3: 5′-ACTTTTATATACAAAATAACTAAATCTCGAGATGGGCGAAAAATTAAAAC G-3′, MuC4mt-R was set forth in SEQ ID NO:4: 5′-ACTAGTCGCAATTGCCGCGGCTCGAGCTACAGAGAAGGTAGAGAAT-3′.

The PCR reaction was conducted according to the PrimeSTAR HS DNA Polymerase (Takara) instruction. The reaction conditions were as follows: denaturing at 95° C. for 3 min, followed by cycles of denaturing at 95° C. for 30 seconds, annealing at 55° C. for 30 seconds, and extending at 72° C. for 1 min. After a total of 30 cycles, extending at 72° C. for 10 minutes, then cooling to 4° C. for 5 minutes. 1113 bp of amplified PCR fragment was obtained and purified. The purified fragment was inserted into the Xhol I endonuclease treated vector pMAT1552, using one-step cloning technology. The ligation product was mixed with Escherichia coli Top10 competent cells and then the mixture was transformed by heat shock. the transformed product was added into 1 ml of LB liquid medium (peptone 10 g/L, yeast extract 5 g/L, NaCl 10 g/L), incubated at 37° C. for 1 h and then coated on LB medium plate containing 100 mg/L ampicillin (peptone 10 g/L, yeast extract 5 g/L, NaCl 10 g/L, agar 1.5%). After cultured at 37° C. overnight, the colonies were selected and inoculated into LB liquid medium. The plasmids were extracted and sequenced after 8˜10 hours, the plasmids with correct sequence were named pMAT1552-C4mt.

### Example 3: Preparation of M. circinelloides Protoplasts

The spores of Mucor circinelloides Mu402 strain were inoculated onto plates of YPG medium (yeast extract 3 g/L, peptone 10 g/L, glucose 20 g/L, leucine 20 μg/mL, uracil 200 μg/mL, pH 4.5), and cultured at 28° C. for 1 day. The monoclonal hyphae were spot inoculated onto the plates of YPG medium and cultivated at 28° C. for 3˜4 days to obtain the well-grown spores. The plates with well-grown spores were taken, 5-6 mL of YPG medium was added to each plate, the spores were scraped with a sterilized coating rod, the spore suspension was collected into a sterilized 50 mL centrifuge tube, the concentration of the spores in the suspension was calculated by using a blood cell counting plate, and the concentration of spores was adjusted to 1×10⁷/ml by using YPG with pH 4.5. 12.5 mL of the above spore suspension was taken into a sterilized 250 mL conical flask and placed in a refrigerator at 4° C. overnight to make the spores fully absorb water and expand. The conical flask was kept on a shaker at 30° C. and 250 rpm until the spores germinated. The spores were washed twice by using 5 mL of PS buffer with pH 6.5 (18.22 g of sorbitol and 20 mL of PBS buffer (NaCl 137 mM, KCl 2.7 mM, Na₂HPO₄ 10 mM, KH₂PO₄ 2 mM)) after centrifugation at 1100 rpm, and the medium was washed away. The cells were resuspended in 5 ml of PS buffer, the lyase at a final concentration of 4 mg/ml and a chitosanase at 0.06 U/ml was added, and incubated for 90 min in a shaker at 30° C. and 60 rpm to remove cell walls. The products after incubation were centrifugated at 100×g, and then washed twice with 0.5 M sorbitol pre-cooled at 4° C., 800 μL of 0.5 M sorbitol was added and gently blew and suctioned to resuspend the precipitate to obtain protoplasts, and the protoplasts were sub packaged in 100 μL/tubes for use.

### Example 4: Construction of Recombinant Strain Mu-C4mt

100 μL of the prepared protoplasts were taken to mix with 1 μg of plasmid pMAT1552-C4mt or pMAT1552, and the mixture was transformed by electro transformation. 1 mL of pre-chilled YPGS (sorbitol 0.5 mol/L, yeast extract 3 g/L, peptone 10 g/L, glucose 20 g/L) was added immediately after the electric shock, incubated at 26° C. and 100 rpm for 1 h, YPGS was removed by centrifugation at 100×g, the precipitate was resuspended by using YNBS (sorbitol 91.1 g/L, glutamic acid 1.5 g/L, (NH₄)₂SO₄ 1.5 g IL, Yeast Nitrogen Base 0.5 g/L, glucose 10 g/L, adjusted pH to 4.5, thiamine and nicotinic acid were added to a final concentration of 1 μg/mL after sterilization), and then uniformly coated on the MMC selective medium (Casamino acid 10 g/L, Yeast Nitrogen Base 0.5 g/L, glucose 20 g/L, agar 15 g/L, adjusted to pH 3.2, thiamine and nicotinic acid were added to a final concentration of 1 μg/mL after sterilization), cultured avoid light at 28° C. for 3˜4 days. Eight single colonies of hyphae growing on the selective plates were randomly picked up and transferred to a new MMC plate, cultured at 28° C. for 2˜3 days to collect spores, and about 200 to 300 spores were respectively inoculated on MMC plates and MMC plates containing uracil, cultured at 28° C. for 2˜3 days to perform colony count, repeated the above screening steps until the growing number of the spores on the two plates was the same, indicating that stable genetic transformants were obtained. The stable genetic transformants hyphae were cultured on YPG medium plated at 30° C. for 5˜7 days, and then spores were collected, the spore concentration was adjusted to 1×10⁷ cells/mL, and the spores were stored in 30% glycerol tube at −80° C. Finally, the recombinant strain Mu-C4mt of M. circinelloides and the control strain Mc1552 were obtained. The remaining fungal cells cultured in the shake flask after coating were separated by vacuum filtration with a Buchner funnel, and the genomic DNA of M. circinelloides was extracted (by referring to the instructions of the plant rapid DNA extraction kit), the genomic DNA was used as a template and 1552-F and 1552-R were used as the primer (the pair of primers were respectively at a position 600 bp upstream and downstream of the inserted target gene site locus in the plasmid) for PCR identification.

(set forth in SEQ ID NO: 5)
1552-F: 5′-CCTCGGCGTCATGATGTTTTTGTGTACCT-3′,
(set forth in SEQ ID NO: 6)
1552-R: 5′-GGGATGTCTGCTGCTACCATGTCTCAT-3′.

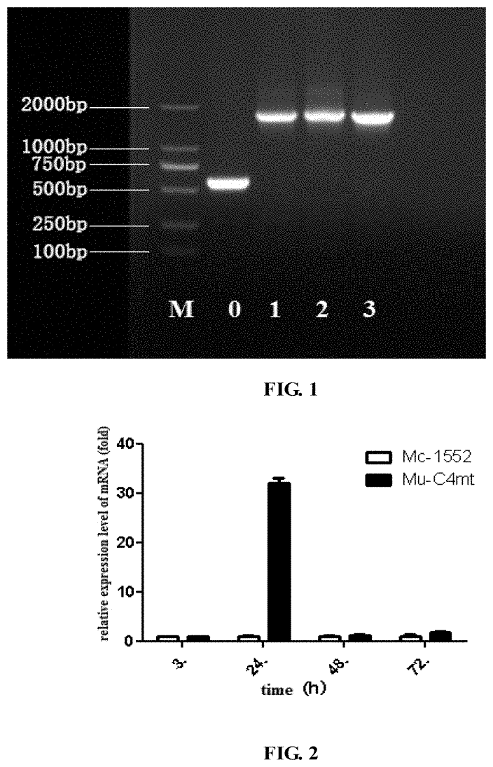

The reaction system and amplification conditions were as follows: denaturing at 95° C. for 3 min, denaturing at 95° C. for 30 seconds, annealing at 60° C. for 30 seconds, and extending at 72° C. for 2 min. 30 cycles, and final extending at 72° C. for 10 minutes. The PCR identification result was as shown in FIG. 1. The fragment obtained by the recombinant strain Mu-C4mt of M. circinelloides is 1713 bp, while the fragment obtained by the corresponding position of the control strain Mc1552 is 600 bp, indicating that the plasmid has been successfully transformed into M. circinelloides.

### Example 5: Fermentation of the Recombinant Strain Mu-C4mt of M. circinelloides and Preparation of Samples to be Tested

The recombinant strain Mu-C4mt of M. circinelloides was cultured on Kendrick medium in a 2 L fermentor. The fermentation conditions were 28° C., 700 rpm, air intake 1 v/v min⁻¹, and pH maintained at 6.0. The whole fermentation broth sample was collected according to the oil production law of M. circinelloides, and vacuum filtrated with a Buchner funnel, the fermentation broth and the mycelium were separated, the fermentation broth was collected and stored at −20° C. to reserve, the mycelium were washed for 3 times with distilled water, then lyophilized to reserve.

### Example 6: Determination of the Expression Level of C4-Dicarboxylic Acid Transporter Gene (C4mt)

The mRNA of 3, 24, 48, 27 h fermented samples was extracted according to the Trizol instruction manual, and the mRNA was reversed to cDNA by using the ReverTra Ace qPCRRT Kit (Roche), the expression level of the C4-dicarboxylic acid transporter was determined by using the RT-qPCR method, the data were processed by using the professional method, SYBR Green Realtime PCR Master Mix (Roche) was used as the kit in the determination process, and the amplification primer sequences were as follows:

(set forth in SEQ ID NO: 7)
WJ11-c4mt-F: 5′-TTATTTTTATCGTTTGGTGGTACACAAA
CTGC-3′,
(set forth in SEQ ID NO: 8)
WJ11-c4mt-R 5′-GTAACCCCACAGAAATAGAGCCATAAGG-3′,

Actin was the reference gene, and the amplification primer sequences were as follows:

(set forth in SEQ ID NO: 9)
actin-F 5′-GATGAAGCCCAATCCAAGA-3′,
(set forth in SEQ ID NO: 10)
actin-R 5′-TTCTCACGGTTGGACTTGG-3′.

The amplification conditions were as follows: preheating at 95° C. for 10 min, then 95° C. for 30s, 59° C. for 10s, and 72° C. for 30s (45 cycles). The result of C4mt gene expression was as shown in FIG. 2. In Mu-C4mt, the C4mt gene was successfully expressed, and the gene expression level was decreased after 24 hours, but the gene expression level was still at a higher level compared with the control.

### Example 7: Fatty Acid Composition and Content Determination of Recombinant Strain Mu-C4mt of M. circinelloides

The oil in dry microbial cells of recombinant strain Mu-C4mt was extracted with an organic solvent, using a wall breaking method which combining acid treatment and repeated freezing and thawing, the method was appropriately modified according to (Folch J, Lees M, Sloane-Stanley G, et al. A simple method for the isolation and purification of total lipids from animal tissues. BiolChem, 1957, 226, 497-509), the specific method was as follows:

1) After grinding the freeze-dried cells, 20 mg dry weight of cells was weighed into a 5 mL glass bottle, and 2 mL of 4 M hydrochloric acid added;

2) The mixture was placed in a water bath at 80° C. for 1 h, at −80° C. for 15 min, repeated once;

3) After returning to room temperature, 1 mL of methanol and 1 mL of chloroform were added, and 100 μL of internal standard C15:0 with a concentration of 2.02 μg/μL was added by using a micro-injector:

4) The mixed solution obtained above was put in a whirlpool mixer for rotation extraction for 0.5 h, centrifuged at 3000 rpm for 3 min, and the chloroform layer was collected in a new 5 mL glass bottle;

5) 1 mL of chloroform was added to the original glass bottle again, repeated the process of 4) and the chloroform layers were combined;

6) The combined chloroform layer solution was blow-dried with nitrogen;

7) 1 mL of 10% methanol solution of hydrochloric acid was added, the added mixed solution was placed in a water bath at 60° C. for 3 hours, and oscillated for 30 seconds every half an hour during the period;

8) 2 mL of n-hexane and 1 mL of saturated NaCl solution were added after cooling to room temperature, the above solution was mixed evenly by vortex and oscillation, and centrifuged at 4000 rpm for 3 min. 1 mL of n-hexane layer was aspirated and transferred to a gas-phase bottle to obtain a fatty acid methyl ester solution.

Commercial fatty acid methyl ester standards (mixed standard of 37 kinds of fatty acid methyl esters) was used as a standard sample to analyze the fatty acid methyl ester by gas chromatography. The gas chromatograph was Agilent GC-6890N in America, the measurement conditions were as follows: gas chromatographic conditions: Splitless injecting samples, the chromatographic column was DM-FFAP (30 m×0.32 mm, 0.22 μm), a flame ionization detector, nitrogen was carrier gas, the temperature of a gasification chamber and the temperature of the detector were both 250° C., and the injection volume was 1 μL. Temperature rising procedure: the initial temperature was 80° C., firstly, the temperature was raised to 200° C. at a heating rate of 8° C./min, then the temperature was raised to 205° (C at a heating rate of 1° C./min, and finally the temperature was raised to 240° C. at a heating rate of 4° C./min, kept for 5 min. Pentadecanoic acid (C15:0) was taken as a reference, the peak area of each fatty acid composition was recorded, and the total fatty acid content was calculated. The results were shown in Table 1. The fatty acid composition of the intracellular of the over-expression strain Mu-C4mt had little change, but the total fatty acid content of the over-expression strain Mu-C4mt was increased by 25.30%, and the intracellular lipid content could reach up to 16.34% of the total fatty acid.

TABLE 1
Oil content of control strain and Mu-C4mt over-expression strain
by fermentation culture

| | Fermentation time (h) | | | | | | | |
|---|---|---|---|---|---|---|---|---|
| strain | 12 | 24 | 36 | 48 | 60 | 72 | 84 | 96 |
| Mu-C4mt | 8.28 | 11.45 | 12.85 | 13.86 | 14.36 | 14.95 | 15.19 | 16.34 |
| Mc1552 | 5.90 | 9.31 | 11.86 | 12.39 | 12.40 | 12.86 | 13.21 | 13.04 |

From this, it can be determined that the protein encoded by the 00018.48 gene of M. circinelloides WJ11 is a C4-dicarboxylic acid transporter, and the protein is successfully expressed in the recombinant strain Mu-C4mt, the protein participates in the oil synthesis process of M. circinelloides, and the intracellular oil production of the strain may effectively increase by over-expressing the transporter.

The above description of the embodiments is only used to help understand the method and core idea of the present application, it should be understood by those skilled in the art that, without departing from the principle of the present application, several improvements and modifications can be made, and these improvements and modifications also should be regarded as the protection scope of the present application fall into the scope of the present application. Various modifications to these embodiments will be obvious to those skilled in the art, and the general principles defined herein may be implemented in other embodiments without departing from the spirit or scope of the present application. Therefore, the present application will not be limited to the embodiments shown in this document, but should conform to the widest scope consistent with the principles and novel features disclosed herein.

1. A C4-dicarboxylic acid transporter suitable for increasing oil yield of Mucor circinelloides,
wherein the C4-dicarboxylic acid transporter comprises the amino acid sequence of SEQ ID NO:2.

2. (canceled)

3. A recombinant vector, comprising:
a C4mt gene encoding the C4-dicarboxylic acid transporter of claim 1,
wherein the C4mt gene is inserted into a base vector pMAT1552, and
wherein the C4mt gene comprises the nucleotide sequence of SEQ ID NO:1.

4. The recombinant vector of claim 3, wherein the recombinant vector capable of expressing the C4-dicarboxylic acid transporter of M. circinelloides.

5. (canceled)

6. A transformant, comprising the recombinant vector of claim 3.

7. The transformant of claim 6, comprising the recombinant vector capable of expressing the C4-dicarboxylic acid transporter of M. circinelloides.

8. The transformant of claim 6, wherein M. circinelloides is a host strain of the recombinant vector.

9. The transformant of claim 8, wherein the M. circinelloides is a M. circinelloides deficient strain Mu402.

10. (canceled)

11. The recombinant vector of claim 3, which is suitable for gene expression in M. circinelloides.

12. The recombinant vector of claim 6, wherein the recombinant vector is suitable for gene expression in M. circinelloides.
